(19) United States
(12) Patent Application Publication
(10) Pub. No.: US 2021/0077364 A1
(43) Pub. Date: Mar. 18, 2021

(54) THICK WATER-BASED COSMETIC

(71) Applicant: Shiseido Company, Ltd., Chuo-ku, Tokyo (JP)

(72) Inventors: Atsushi SOGABE, Kanagawa (JP); Makoto UYAMA, Kanagawa (JP); Naoki SAITO, Kanagawa (JP); Yuka HIROSE, Kanagawa (JP); Kohei MATSUMORI, Kanagawa (JP)

(73) Assignee: Shiseido Company, Ltd., Chuo-ku, Tokyo (JP)

(21) Appl. No.: 17/046,860

(22) PCT Filed: Mar. 20, 2019
(86) PCT No.: PCT/JP2019/011882

§ 371 Date: Oct. 12, 2020

(30) Foreign Application Priority Data
Apr. 16, 2018 (JP) 2018-078399

(51) Int. Cl. A61K 8/04; A61K 8/87; A61K 8/06; A61Q 19/00

(52) U.S. Cl. CPC A61K 8/042; A61K 8/87; A61K 2800/41; A61Q 19/00; A61K 8/062

(57) ABSTRACT

The invention provides a thick water-based cosmetic which can impart a novel usage feeling

The thick water-based cosmetic of the present invention has a viscoelastic ratio of 10 or more and a viscosity of 500 mPa·s or less at a shear rate of 1/s, wherein when concentrated by a factor of five by drying to remove moisture, the thick water-based cosmetic changes to a gel-like form having a viscoelastic ratio of 0.5 or less and a viscosity of 70000 mPa·s or more at a shear rate of 1/s.

### FIELD

The present invention relates to a thick water-based cosmetic.

### BACKGROUND

Recently, in the field of cosmetics, various cosmetics have been proposed. For example, there are provided cosmetics that can impart a silky feeling, and gel-like cosmetics which can exhibit a moisturizing effect, a thickness feeling, an adherence feeling, etc.

Patent Literature 1 discloses an elastic gel-like composition with a unique jiggly feeling, in which a hydrophobically-modified polyether urethane is contained in an oil-in-water emulsion having oil droplets having an average particle diameter of 150 nm or less.

Patent Literature 2 describes a cosmetic which imparts an excellent silky feeling and which comprises (A) a silicone resin powder, (B) an amphoteric polymer, (C) a nonionic surfactant, and (D) water, wherein the ratio [(A)/(B)] of the (A) silicone resin powder to the (B) amphoteric polymer is 5 to 40.

Patent Literature 3 discloses a gel-like cosmetic having a moisturizing effect, comprising (1) a carboxyvinyl polymer which may have a C₁₀₋₃₀ alkyl group or may have a crosslinked structure, and/or a salt thereof, (2) trehalose which may be sulfated and/or a salt thereof, and (3) a water-absorbing polymer.

Patent Literature 4 discloses a gel-like cosmetic containing a (hydroxyethyl acrylate/acryloyl dimethyl taurine Na) copolymer and a polyhydric alcohol, and further containing glycerin as a polyhydric alcohol, and one or more selected from sorbitol, maltitol, and diglycerin, wherein the gel-like cosmetic has a high thickness feeling and adherence feeling.

### CITATION LIST

### Patent Literature

### [PTL 1] Japanese Unexamined Patent Publication (Kokai) No. 2016-088868

### [PTL 2] Japanese Unexamined Patent Publication (Kokai) No. 2013-112680

### [PTL 3] Japanese Unexamined Patent Publication (Kokai) No. 2005-225769

### [PTL 4] Japanese Unexamined Patent Publication (Kokai) No. 2017-109962

### SUMMARY

### Technical Problem

In the field of cosmetics, conventionally, there has been demand for cosmetics that can impart a silky feeling, gel-like cosmetics that can impart a jiggly feeling, and cosmetics that can impart novel usage feelings different from those of the past.

Thus, the object of the present invention is to provide a thick water-based cosmetic which can impart a novel usage feeling.

### Solution to Problem

### <Aspect 1>

A thick water-based cosmetic having a viscoelastic ratio of 10 or more and a viscosity of 500 mPa·s or less at a shear rate of 1/s, wherein

when concentrated by a factor of five by drying to remove moisture, the thick water-based cosmetic changes to a gel-like form having a viscoelastic ratio of 0.5 or less and a viscosity of 70000 mPa·s or more at a shear rate of 1/s.

### <Aspect 2>

The cosmetic according to Aspect 1, wherein the cosmetic in the gel-like form has a viscoelastic ratio of 0.2 or less and a viscosity of 75000 mPa·s or more at a shear rate of 1/s.

### <Aspect 3>

The cosmetic according to Aspect 1 or 2, wherein the cosmetic in the gel-like form has a viscoelastic ratio of 0.005 or more and a viscosity of 500000 mPa·s or less at a shear rate of 1/s.

### <Aspect 4>

The cosmetic according to any one of Aspects 1 to 3, wherein in the cosmetic, an oil-in-water emulsion containing oil droplets is mixed with a hydrophobically-modified polyether urethane.

### <Aspect 5>

The cosmetic according to Aspect 4, wherein the average particle diameter of the oil droplets is 150 nm or less.

### <Aspect 6>

The cosmetic according to Aspect 4 or 5, wherein the oil droplets contain oil and a surfactant.

### <Aspect 7>

The cosmetic according to any one of Aspects 4 to 6, wherein the hydrophobically-modified polyether urethan is represented by formula 1 below:

Rⁱ—{(O—Rⁱⁱ)ₖ—OCONH—Rⁱⁱⁱ[—NHCOO—(R^{iv}—O)ₚ—R^{v}]ₕ}_{q} formula 1

where Rⁱ, Rⁱⁱ, and R^{iv} each independently represent a C₂₋₄ hydrocarbon group,

Rⁱⁱⁱ represents a C₁₋₁₀ hydrocarbon group optionally having a urethane bond,

R^{v} represents a C₈₋₃₆ hydrocarbon group,

k is an integer of 1 to 500,

p is an integer of 1 to 200,

h is an integer of 1 or more, and

q is an integer of 2 or more.

### <Aspect 8>

The cosmetic according to Aspect 7, wherein the hydrophobically-modified polyether urethane is a polyethylene glycol-decyl tetradeceth-hexamethylene diisocyanate copolymer.

### Advantageous Effects of Invention

According to the present invention, a thick water-based cosmetic which can impart a novel usage feeling, and in particular, a thick water-based cosmetic which has a thick form and which changes to a gel-like form when concentrated, thereby imparting a novel usage feeling can be provided.

### BRIEF DESCRIPTION OF DRAWINGS

FIG. 1(a) is a schematic diagram of a cosmetic according to an embodiment of the present application prior to concentration and (b) is a schematic view of the cosmetic of the embodiment of the present application after concentration.

FIG. 2 is a graph related to the shear viscosities and viscoelastic ratios prior to and after five-fold concentration of the cosmetics of the embodiment of the present invention and a comparative sample.

### DESCRIPTION OF EMBODIMENTS

The embodiments of the present invention will be described in detail below. The present invention is not limited to the following embodiments, and various changes can be made within the scope of the gist of the invention.

The thick water-based cosmetic of the present invention, when in the thick form prior to concentration, has a viscoelastic ratio of 10 or more and a viscosity of 500 mPa·s or less at a shear rate of 1/s, and when in the gel-like form after five-fold concentration, has a viscoelastic ratio of 0.5 or less and a viscosity of 70000 mPa·s or more at a shear rate of 1/s.

If a conventional cosmetic is highly concentrated to 10-fold or more or 20-fold or more, the viscosity and viscoelastic ratio after concentration may be satisfied. However, there have been no cosmetics exhibiting such a morphological change by approximately five-fold concentration, i.e., by such low-fold concentration. When such cosmetics are applied to the skin, the morphological change can be actually felt.

In the present invention, “five-fold concentration” defines the measurement conditions of the viscosity and viscoelastic ratio of a concentrated cosmetic, and does not mean that the cosmetic satisfies the viscosity and viscoelastic ratio described above only at five-fold concentration. In other words, as long as the above-mentioned viscosity and viscoelastic ratio are satisfied at five-fold concentration, the cosmetic may further satisfy the above-mentioned viscosity and viscoelastic ratio at, for example, two- or ten-fold concentration.

The thick water-based cosmetic of the present invention may be any composition as long as it is a cosmetic exhibiting the viscosity and viscoelastic ratio described above, and may be, for example, a cosmetic comprising a microemulsion, a nanoemulsion, or an oil-in-water emulsion having oil droplets such as high-pressure emulsified particles, a cosmetic comprising a polyion complex, or a cosmetic comprising a vesicle. These cosmetics may appropriately use a material such as a hydrophobically-modified polyether urethane, which is shown below.

Though not limited by theory, it is considered that the operating principle by which the thick water-based cosmetic is changed into a gel state by concentration is as follows.

For example, in the case of a cosmetic obtained by mixing a hydrophobically-modified polyether urethane into an oil-in-water emulsion having oil droplets, it is considered that the oil droplets and the hydrophobically-modified polyether urethane are dispersed in water so that at least a part of the hydrophobically-modified polyether urethane is interposed between adjacent oil droplets.

As a result, such a cosmetic has a large quantity of water contained in the cosmetic prior to concentration, and is in a thick state, but as such a cosmetic is concentrated, crosslinking points are formed between the oil droplets and hydrophobically-modified polyether urethane, and a linked network structure is developed, whereby moisture is easily retained, a gel is formed, and a hydrophobically-modified polyether urethane exhibits cushioning action between oil droplets. As described above, in the case of such a cosmetic, since the thickening effect by the hydrophobically-modified polyether urethane itself and the thickening effect accompanied by the development of the network structure due to the formation of the crosslinking points between the oil droplets and the hydrophobically-modified polyether urethane can be synergistically exhibited, it is possible to change from a smooth usage feeling in a thick state to an elastic gel-like jiggly usage feeling after concentration.

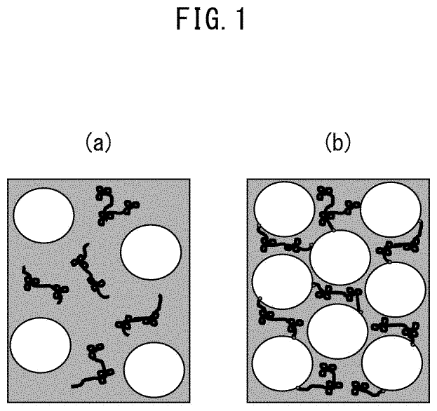

More specifically, for example, immediately after the cosmetic described above as an example is applied to the skin, the amount of moisture therein is large, and as shown in (a) in FIG. 1, the formation of a network structure is insufficient, and thus, it has a low viscosity and a thick, smooth feeling. However, as the cosmetic dries and is concentrated, the distance between the oil droplets and the hydrophobically-modified polyether urethane located between the oil droplets becomes increasingly small, such that crosslinking points are more easily formed with the oil droplets, as shown in (b) of FIG. 1, and a network structure in which the oil droplets and the hydrophobically-modified polyether urethane are connected is also easily formed. As a result, the viscosity and the ability to retain moisture increase, as well as the elasticity associated with the network structure is also exhibited, whereby the cosmetic is considered to change into a gel-like form which exhibits an elastic jiggly usage feeling.

Among oil droplets, in particular, oil droplets having an average particle diameter of 150 nm or less emulsified under high pressure exhibit a tendency to resist breaking down, even if a hydrophobic portion of the hydrophobically-modified polyether urethane forms a crosslinking point with an oil droplet. It is believed that this is because the surfaces of such oil droplets are in a relatively stable state, such as a solid film.

In addition, for example, when an attempt is made to develop such an elastic gel-like form using only the hydrophobically-modified polyether urethane, the hydrophobically-modified polyether urethane must be used in larger quantity than in cosmetics in which the hydrophobically-modified polyether urethane is mixed into an oil-in-water type emulsion having oil droplets. As a result, it is considered that an increase in cost and an increase in stickiness derived from a hydrophobically-modified polyether urethane are brought about. Conversely, for example, for a cosmetic obtained by mixing a hydrophobically-modified polyether urethane into an oil-in-water emulsion having oil droplets, cost can be further reduced as compared with a system in which only a hydrophobically-modified polyether urethane is used, and a moist feeling while reducing stickiness derived from the hydrophobically-modified polyether urethane can be achieved.

The definitions of the terms of the present invention are as follows.

In the present invention, the term “thick” is intended to indicate a state of a liquid having a lower viscosity than general gel-like cosmetics, but having a higher viscosity than water, such as ion-exchanged water. Therefore, the thick water-based cosmetic of the present invention has a different form from general gel-like cosmetics and ion-exchanged water.

The term “thick” in the present invention can encompass cosmetics exhibiting a viscoelastic ratio (tan δ=loss elastic modulus/storage elastic modulus) value of 10 or more in a linear region at a shear rate of 1/s when measured at 32° C. and 1 atm using, for example, an MCR-302 (manufactured by Anton-Parr) as the rheometer and a viscosity of 500 mPa·s or less, 250 mPa·s or less, or 100 mPa·s or less at a shear rate of 1/s.

In the present invention, the term “gel-like” means a state having flexibility like a liquid and having elasticity to the extent that a tendency to return to the original shape when stress is applied is exhibited, and having higher viscosity than a thick state. For example, this term can encompass cosmetics exhibiting a viscoelastic ratio of 0.5 or less, 0.4 or less, 0.3 or less, or 0.2 or less when measured at 32° C. and 1 atm using an MCR-302 (manufactured by Anton-Parr) as the rheometer, and exhibiting a viscosity of 70000 mPa·s or more, 75000 mPa·s or more, or 80000 mPa·s or more at a shear rate of 1/s. The lower limit value of the viscoelastic ratio can be 0.005 or more, 0.006 or more, 0.007 or more, or 0.008 or more, and the upper limit value of the viscosity can be 500000 mPa·s or less, 400000 mPa·s or less, 30000 mPa·s or less, or 200000 mPa·s or less.

In the present invention, the term “crosslinking point” means a site where at least one of the hydrophobic portions of at least one hydrophobically-modified polyether urethane is taken into an oil droplet or adsorbed in the vicinity of the surface of oil droplet, unlike crosslinking points based on polymerization.

### <<Oil-in-Water Emulsion>>

The cosmetic of the present invention may be any cosmetic as long as the viscosity and the viscoelastic ratio prior to and after concentration fall within the specific ranges described above, and is not limited to the following, but may be, for example, an oil-in-water emulsion having oil droplets. Such an oil-in-water emulsion is an emulsified cosmetic in which oil droplets, as a dispersed phase, are dispersed in water, which is a continuous phase.

### <Oil Droplets>

The oil droplets as an oil phase or dispersed phase in the oil-in-water emulsion may include oil and a surfactant, and may optionally also include a higher alcohol.

The oil content in the cosmetic of the present invention is not limited to the following, but can be, for example, 0.5% by mass or more, 1.0% by mass or more, 1.2% by mass or more, or 1.5% by mass or more relative to the total quantity of the cosmetic. The upper limit value of the oil content is not particularly limited, and may be 25% by mass or less, 20% by mass or less, or 18% by mass or less. From the viewpoint of dispersibility or exhibiting an effective action as an oil, the oil content is preferably in the range of 2 to 15% by mass.

The mixing amount of the surfactant and the higher alcohol is not limited to the following. The total mixing amount of the surfactant and the higher alcohol, if present, may be 0.2% by mass or more, 0.5% by mass or more, or 1.0% by mass or more with respect to the aqueous phase, and the upper limit value is not particularly limited, but may be 10% by mass or less, 9% by mass or less, or 8% by mass or less. The oil content relative to the total mixing amount of the surfactant and the higher alcohol, if present, can be 1/3 or more, and the upper limit value is not particularly limited, but may be 5 or less.

From the viewpoint of stability during gelation or the like, the oil-in-water emulsion used in the present invention is preferably an ultrafine emulsion containing oil droplets having an average particle diameter on the order of nanometers. The average particle diameter of such oil droplets can be, for example, 150 nm or less, 140 nm or less, 130 nm or less, 120 nm or less, or 110 nm or less, and when the cosmetic is transparent or translucent, the average particle diameter is preferably 100 nm or less, 90 nm or less, or 80 nm or less. The lower limit of the average particle size is not particularly limited, and may be, for example, 5 nm or more, 10 nm or more, 20 nm or more, or 50 nm or more. The average particle diameter of the oil droplets can be defined as an average value of the diameters of the oil droplets measured optically by a dynamic light scattering method when, for example, the particle shapes of the oil droplets is assumed to be spherical.

An oil-in-water emulsion containing ultrafine oil droplets having an average particle diameter of 150 nm or less (sometimes referred to as an “ultrafine emulsion”) can be prepared by a method such as an agglomeration method or a dispersion method.

The agglomeration method is a method for preparing colloids which utilizes interfacial chemical characteristics, and is a method in which a uniformly dissolved state is made into a supersaturated state by a certain means, whereby a dispersed phase appears. As specific examples of such a method, an HLB temperature emulsification method, a phase inversion emulsification method, a non-aqueous emulsification method, a D-phase emulsification method, and a liquid crystal emulsification method are known.

The dispersion method is a method in which a mass of a dispersed phase is finely divided by force. The dispersion method preferably used in the present invention is a dispersion method by high pressure emulsification, as described in Japanese Patent No. 3398171. “High-pressure emulsification” as used herein is a method of preliminarily emulsifying an aqueous phase component and an oil phase component with a homomixer or the like to obtain an emulsion having fine emulsified particles by high shearing force using, for example, a high pressure homogenizer under high pressure.

### (Oil)

The oil may be any of a liquid oil, a solid oil, and a semi-solid oil. For example, the oil may be selected from a liquid oil such as avocado oil, camellia oil, turtle oil, macadamia nut oil, corn oil, mink oil, olive oil, rapeseed oil, egg yolk oil, sesame oil, persic oil, wheat germ oil, sasanqua oil, castor oil, linseed oil, safflower oil, cottonseed oil, evening primrose oil, eno oil, soybean oil, peanut oil, tea seed oil, kaya oil, rice bran oil, Cina Kiri oil, Japanese Kiri oil, jojoba oil, germ oil, triglycerin, glyceryl trioctanoate, glyceryl triisopalmitate, and hydrogenated polydecene; a solid oil such as cocoa butter, coconut oil, horse fat, hardened coconut oil, palm oil, beef tallow, sheep fat, hardened beef tallow, palm kernel oil, pork fat, beef bone fat, Japan wax kernel oil, hydrogenated oil, beef foot oil, Japan wax, and hydrogenated castor oil; a wax such as beeswax, candelilla wax, cotton wax, carnauba wax, bayberry wax, insect wax, whale wax, montan wax, nuka wax, lanolin, cabox wax, lanolin acetate, liquid lanolin, sugar cane wax, lanolin fatty acid isopropyl, hexyl laurate, reduced lanolin, jojoba wax, hard lanolin, shellac wax, POE lanolin alcohol ether, POE lanolin alcohol acetate, lanolin fatty acid polyethylene glycol, and POE hydrogenated lanolin alcohol ether; a hydrocarbon such as olefin oligomer, isododecane, isohexadecane, liquid paraffin, ozokerite, squalene, pristane, paraffin, ceresin, squalane, petrolatum, and microcrystalline wax; a synthetic ester such as isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexylate, dipentaerythritol fatty acid ester, N-alkyl glycol monoisostearate, neopentyl glycol dicaprate, diisostearyl malate, glycerin di-2-heptylundecanoate, trimethylolpropane tri-2-ethylhexylate, trimethylolpropane triisostearate, pentaerythritol tetra-2-ethylhexylate, glycerin tri-2-ethylhexylate, trimethylolpropane triisostearate, cetyl-2-ethylhexanoate, 2-ethylhexyl palmitate, glycerin trimyristate, glyceride tri-2-heptylundecanoate, fatty acid methyl ester of castor oil, oleic acid oil, cetostearyl alcohol, acetoglyceride, 2-heptylundecyl palmitate, diisopropyl adipate, N-lauroyl-L-glutamic acid-2-octyldodecyl ester, di-2-heptylundecyl adipate, ethyl laurate, di-2-ethylhexyl separate, 2-hexyldecyl myristate, 2-hexyldecyl palmitate, 2-hexyldecyl adipate, diisopropyl sebacylate, 2-ethylhexyl succinate, ethyl acetate, butyl acetate, amyl acetate, and triethyl citrate; a silicone oil such as dimethylpolysiloxane and methylphenylpolysiloxane, a perfluorocarbon or perfluoropolyethers such as perfluorodecalin, perfluorohexane, and triperfluoro-n-butylamine; a vitamin such as vitamin A and its derivatives, vitamin D and its derivatives, vitamin E and its derivatives, and vitamin K and its derivatives; a sterol; and natural and synthetic fragrances.

### (Surfactant)

The surfactant is not particularly limited, but an anionic, cationic or zwitterionic surfactant or a nonionic surfactant can be used. Among these, anionic and cationic ionic surfactants are preferred.

### (Higher Alcohol)

For example, in the case of a microemulsion prepared by high-pressure emulsification described above, the oil droplets may comprise a surfactant, a higher alcohol and an oil selected from those capable of forming a gel at room temperature or higher in a system of a surfactant, a higher alcohol, and water. In particular, it is preferable that a substantial total amount of the higher alcohol and surfactant be present at the oil droplet interface. The gel is preferably α-type from the viewpoint of stability, and the transition temperature of the gel is preferably 60° C. or more. As such a higher alcohol, higher alcohols having a carbon chain length of 16 or more are preferred. Specific examples thereof include linear or branched higher alcohols such as lauryl alcohol, cetyl alcohol, stearyl alcohol, behenyl alcohol, myristyl alcohol, oleyl alcohol, cetostearyl alcohol, monostearyl glycerene ether (batyl alcohol), 2-decyltetradecinol, lanolin alcohol, cholesterol, hexyldodecanol, isostearyl alcohol, and octyldodecanol.

Combinations of higher alcohols and surfactants (expressed below in the form of higher alcohol-surfactant) include, but are not limited to, behenyl alcohol (higher alcohol)-behenic acid soap (surfactant), stearyl alcohol (higher alcohol)-stearate soap (surfactant), stearyl alcohol (higher alcohol)-sodium cetyl sulfate (surfactant), behenyl alcohol (higher alcohol)-behenyltrimethylammonium chloride (surfactant), behenyl alcohol (higher alcohol)-stearyl trimethylammonium chloride (surfactant), and behenyl alcohol and/or stearyl alcohol (higher alcohol)-sodium stearoyl glutamate (surfactant).

### <<Hydrophobically-Modified Polyether Urethane>>

The hydrophobically-modified polyether urethane of the present invention is a material also referred to as an associative thickener or an associative polymer, is not limited to the following, and can be a hydrophobically-modified polyether urethane represented by the following formula 1:

Rⁱ—{(O—Rⁱⁱ)ₖ—OCONH—Rⁱⁱⁱ[—NHCOO—(R^{iv}—O)ₚ—R^{v}]ₕ}_{q} formula 1

Preferred examples of hydrophobically-modified polyether urethanes include polyethylene glycol-decyltetradeceth-hexamethylene diisocyanate copolymers. Particularly preferred examples include the hydrophobically-modified polyether urethane known by the INCI names “(PEG-240/decyltetradeceth-20/HDI) copolymer (PEG-240/HDI COPOLYMER BISDECYLTE TRADECETH-20 ETHER).” This copolymer is commercially available from ADEKA Co., Ltd., under the trade name “Adekanol GT-700.”

In formula 1, Rⁱ, Rⁱⁱ, and R^{iv} are each independently a C₂₋₄ hydrocarbon group, and are preferably an C₂₋₄ alkyl group or alkylene group. Rⁱⁱⁱ represents a C₁₋₁₀ hydrocarbon group optionally having a urethane bond. R^{v} represents a C₈₋₃₆, preferably a C₁₂₋₂₄, hydrocarbon group. k is an integer from 1 to 500, preferably an integer from 100 to 300. p is an integer from 1 to 200, preferably an integer from 10 to 100. h is an integer of 1 or more, preferably 1. q is an integer of 2 or more, and is preferably 2.

The hydrophobically-modified polyether urethane represented by formula 1 can be obtained by reacting, for example, one or more of the polyether polyols represented by Rⁱ—[(O—Rⁱⁱ)ₖ—OH]_{q}, one or more of the polyisocyanates represented by Rⁱⁱⁱ—(NCO)ₕ₊₁, or one or more of the polyether monoalcohols represented by HO—(R^{iv}—O)ₚ—R^{v}. Rⁱ, Rⁱⁱ, Rⁱⁱⁱ, R^{v}, k, p, h, and q as used herein are as defined above.

In this production method, Rⁱ to R^{v} in formula 1 are determined in accordance with Rⁱ—[(O—Rⁱⁱ)ₖ—OH]_{q}, Rⁱⁱⁱ—(NCO)ₕ+₁, and HO—(R^{iv}—O)ₚ—R^{v} as raw materials. Though the mixing ratio of these three components is not particularly limited, it is preferable that the ratio of the isocyanate groups derived from the polyisocyanate to the hydroxyl groups derived from the polyether polyol and the polyether monoalcohol be in the range of NCO/OH=0.8:1 to 1.4:1.

<Polyether Polyol Represented by Rⁱ—[(O—Rⁱⁱ)ₖ—OH]_{q}>

The polyether polyol represented by Rⁱ—[(O—Rⁱⁱ)ₖ—OH]_{q} can be obtained by addition polymerization of an alkylene oxide such as ethylene oxide, propylene oxide, butylene oxide, epichlorohydrin, or styrene oxide to a q-valent polyol.

Polyols having a valence of 2 to 8 are preferable, and examples thereof include divalent alcohols such as ethylene glycol, propylene glycol, butylene glycol, hexamethylene glycol, and neopentyl glycol; trivalent alcohols such as glycerin, trioxyisobutane, 1,2,3-butanetriol, 1,2,3-pentatriol, 2-methyl-1,2,3-propanetriol, 2-methyl-2,3,4-butanetriol, 2-ethyl-1,2,3-butanetriol, 2,3,4-pentanetriol, 2,3,4-hexanetriol, 4-propyl-3,4,5-heptanetriol, 2,4-dimethyl-2,3,4-pentanetriol, pentamethylglycerin, pentaglycerin, 1,2,4-butanetriol, 1,2,4-pentanetriol, trimethylolethane, and trimethylolpropane; tetravalent alcohols such as pentaerythritol, 1,2,3,4-pentanetetrol, 2,3,4,5-hexanetetrol, 1,2,4,5-pentanetetrol, and 1,3,4,5-hexanetetrol; pentavalent alcohols such as adnit, arabite, and xylit; hexavalent alcohols such as dipentaerythritol, sorbit, mannite, and idit; and octavalent alcohols such as sucrose.

Rⁱⁱ is determined in accordance with the alkylene oxide or styrene oxide to be added. C₂₋₄ alkylene oxides or styrene oxides are preferable because they are particularly readily available and can exhibit excellent effects. The alkylene oxide or styrene oxide to be added may be a homopolymer or a random or block polymer of two or more types. The method of addition may be a conventional method. The degree of polymerization k is an integer from 1 to 500. The ratio of ethylene groups in Rⁱⁱ is preferably from 50 to 100% by mass relative to the total Rⁱⁱ.

The molecular weight of Rⁱ—[(O—Rⁱⁱ)ₖ—OH]_{q} is preferably from 500 to 100000, and more preferably from 1000 to 50000.

<Polyisocyanate Represented by Rⁱⁱⁱ—(NCO)ₕ₊₁>

The polyisocyanate represented by Rⁱⁱⁱ—(NCO)ₕ₊₁ is not particularly limited as long as it has two or more isocyanate groups in the molecule thereof, and examples thereof include aliphatic diisocyanates, aromatic diisocyanates, alicyclic diisocyanates, biphenyldiisocyanate, and di-, tri-, or tetra-isocyanates of phenylmethane.

Examples of aliphatic diisocyanates include methylene diisocyanate, dimethylene diisocyanate, trimethylene diisocyanate, tetramethylene diisocyanate, pentamethylene diisocyanate, hexamethylene diisocyanate, dipropyl ether diisocyanate, 2,2-dimethylpentane diisocyanate, 3-methoxyhexane diisocyanate, octamethylene diisocyanate, 2,2,4-trimethylpentane diisocyanate, nonamethylene diisocyanate, decamethylene diisocyanate, 3-butoxyhexane diisocyanate, 1,4-butylene glycol dipropyl ether diisocyanate, thiodihexyl diisocyanate, metaxylylene diisocyanate, paraxylylene diisocyanate, and tetramethylxylylene diisocyanate.

Examples of aromatic diisocyanates include metaphenylene diisocyanate, paraphenylene diisocyanate, 2,4-tolylene diisocyanate, 2,6-tolylene diisocyanate, dimethylbenzene diisocyanate, ethylbenzene diisocyanate, isopropylbenzene diisocyanate, tolidine diisocyanate, 1,4-naphthalene diisocyanate, 1,5-naphthalene diisocyanate, 2,6-naphthalene diisocyanate, and 2,7-naphthalene diisocyanate.

Examples of alicyclic diisocyanates include hydrogenated xylylene diisocyanate and isophorone diisocyanate.

Examples of biphenyl diisocyanates include biphenyl diisocyanate, 3,3′-dimethylbiphenyl diisocyanate, and 3,3′-dimethoxybiphenyl diisocyanate.

Examples of diisocyanates of phenylmethane include diphenylmethane-4,4′-diisocyanate, 2,2′-dimethyldiphenylmethane-4,4′-diisocyanate, diphenyldimethylmethane-4,4′-diisocyanate, 2,5,2′,5′-tetramethyldiphenylmethane-4,4′-diisocyanate, cyclohexylbis(4-isocyontophenyl)methane, 3,3′-dimethoxydiphenylmethane-4,4′-diisocyanate, 4,4′-dimethoxydiphenylmethane-3,3′-diisocyanate, 4,4′-diethoxydiphenylmethane-3,3′-diisocyanate, 2,2′-dimethyl-5,5′-dimethoxydiphenylmethane-4,4′-diisocyanate, 3,3′-dichlorodiphenyldimethylmethane-4,4′-dii socyanate, and benzophenone-3,3′-diisocyanate.

Examples of triisocyanates of phenylmethane include 1-methylbenzene-2,4,6-triisocyanate, 1,3,5-trimethylbenzene-2,4,6-triisocyanate, 1,3,7-naphthalenetriisocyanate, biphenyl-2,4,4′-triisocyanate, diphenylmethane-2,4,4′-triisocyanate, 3-methyldiphenylmethane-4,6′,4′-triisocyanate, triphenylmethane-4,4′,4″-triisocyanate, 1,6,11-undecane triisocyanate, 1,8-diisocyanate-4-isocyanatomethyloctane, 1,3,6-hexamethylene triisocyanate, bicycloheptane triisocyanate, and tris(isocyanate phenyl) thiophosphate.

These polyisocyanate compounds may be used in the form of trimers based on dimers and isocyanurate bonds, or may be used as a biuret by reacting with an amine.

These polyisocyanate compounds and polyisocyanates having a urethane bond obtained by reacting a polyol can also be used. Polyols having a valence of 2 to 8 are preferred, and the polyols described above are preferred. These polyisocyanates having a urethane bond are preferable when a polyisocyanate having a valence of 3 or more is used as Rⁱⁱⁱ—(NCO)ₕ₊₁.

<Polyether Mono-Alcohol Represented by HO—(R^{iv}—O)ₚ—R^{v}>

The polyether mono-alcohol represented by HO—(R^{iv}—O)ₚ—R^{v} is not particularly limited as long as it is a polyether of monovalent alcohol. Such a compound can be obtained by addition polymerization of an alkylene oxide such as ethylene oxide, propylene oxide, butylene oxide, or epichlorohydrin, or a styrene oxide to a monovalent alcohol.

The monovalent alcohols referred to herein are represented by the following formulas I to III

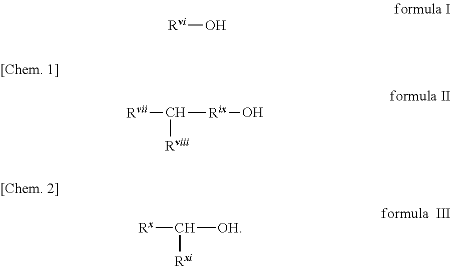

Specifically, R^{v} is a group obtained by removing a hydroxyl group from the monovalent alcohols of the above formulas I to III. In the formulas I to III above, R^{vi}, R^{vii}, R^{viii}, R^{x} and R^{xi} are hydrocarbon groups, for example, alkyl groups, alkenyl groups, alkylaryl groups, cycloalkyl groups, and cycloalkenyl groups.

Examples of alkyl groups include methyl, ethyl, propyl, isopropyl, butyl, isobutyl, tertiary butyl, pentyl, isopentyl, neopentyl, tertiary pentyl, hexyl, heptyl, octyl, 2-ethylhexyl, nonyl, decyl, undecyl, dodecyl, tridecyl, isotridecyl, myristyl, palmityl, stearyl, isostearyl, icosyl, docosyl, tetracocyl, triacontyl, 2-octyldodecyl, 2-dodecylhexadecyl, 2-tetradecyl-octadecyl, and monomethyl-branch-isostearyl.
Examples of alkenyl groups include vinyl, allyl, propenyl, isopropenyl, butenyl, pentenyl, isopentenyl, hexenyl, heptenyl, octenyl, nonenyl, decenyl, undecenyl, dodecenyl, tetradecenyl, and oleyl.

Examples of alkylaryl groups include phenyl, tolyl, xylyl, cumenyl, mesityl, benzyl, phenethyl, styryl, cinnamyl, benzhydryl, trityl, ethylphenyl, propylphenyl, butylphenyl, pentylphenyl, hexylphenyl, heptylphenyl, octylphenyl, nonylphenyl, α-naphthyl, and β-naphthyl groups.

Examples of the cycloalkyl group and the cycloalkenyl group include cyclopentyl, cyclohexyl, cycloheptyl, methylcyclopentyl, methylcyclohexyl, methyl cycloheptyl, cyclopentenyl, cyclohexenyl, cycloheptenyl, methylcyclopentenyl, methyl cyclohexenyl, and methylcycloheptenyl groups.

In Formula II above, R^{ix} is a hydrocarbon group, and is, for example, an alkylene group, an alkenylene group, an alkylarylene group, a cycloalkylene group, or a cycloalkenylene group.

R^{v} is a hydrocarbon group, and thereamong is preferably an alkyl group, and further preferably has 8 to 36 carbon atoms in the sum thereof, and particularly preferably 12 to 24.

The alkylene oxide or styrene oxide to be added may be a homopolymer or a random or block polymer of two or more types. The method of addition may be a conventional method. The degree of polymerization p is an integer of 0 to 1000, preferably an integer of 1 to 200, and more preferably an integer of 10 to 200. The ratio of ethylene group to R^{iv} is preferably in the range of 50 to 100% by mass, and more preferably in the range of 65 to 100% by mass, with respect to the total of R^{iv}.

### (Method for Producing Copolymer Represented by Formula 1)

The copolymer represented by formula 1 above can be produced by, for example, heating at 80 to 90° C. for 1 to 3 hours to react in the same manner as in the general reaction of a polyether and an isocyanate.

When the polyether polyol D represented by Rⁱ—[(O—Rⁱⁱ)ₖ—OH]_{q}, the polyisocyanate E represented by R^{ill}—(NCO)ₕ₊₁ and the polyether mono-alcohol F represented by the HO—(R^{iv}—O)ₚ—R^{v} are reacted, products other than the copolymer having the structure of formula 1 may be by-produced in some cases. For example, when a diisocyanate is used, an F-E-D-E-F copolymer represented by Formula 1 is generated as a main product, but in addition, an F-E-F copolymer, or an F-E-(D-E)ₓ-D-E-F copolymer may be by-produced. In this case, the reaction product can be used in the present invention in the form of a mixture containing the copolymer of formula 1 without separating the copolymer of formula 1.

### (Hydrophobically-Modified Polyether Urethane Mixing Amount)

The amount of the hydrophobically-modified polyether urethane to be mixed in the cosmetic of the present invention may be 0.1% by mass or more, 0.2% by mass or more, 0.3% by mass or more, 0.35% by mass or more, or 0.4% by mass or more with respect to the total amount of the cosmetic, and from the viewpoint of obtaining a novel usage feeling, may be 3% by mass or less, 2% by mass or less, or 1% by mass or less.

### <<Water>>

The amount of water to be mixed in the cosmetic of the present invention is not particularly limited, and is preferably, from the viewpoint of, for example, thick form morphogenicity, 70 to 95% by mass, and more preferably 75 to 90% by mass, with respect to the total amount of the cosmetic.

### <<Other Components>>

In the cosmetic of the present invention, various components can be appropriately mixed, depending on the application of the cosmetic, within a range that does not affect the change in form. Examples of the various components include additives such as lower alcohols, polyhydric alcohols, various extracts, humectants, antioxidants, buffers, preservatives, dyes, perfumes, chelating agents, pH adjusting agents, and ultraviolet absorbers which may be normally mixed into cosmetics. The various components may be mixed depending on the properties thereof, for example, in the case of oil-in-water emulsions, in an aqueous phase as a continuous phase and/or in an oil phase as a dispersed phase, i.e., in an oil droplet.

In the aqueous phase, in addition to a water-soluble agent applicable to medicaments, quasi-medicaments, and cosmetics, any water-based component conventionally used in medicaments and cosmetics may be mixed in an amount within a range that does not affect the change in the form of the cosmetic. In particular, as the water-based component, one or more selected from ethanol and polyols are preferably mixed, from the viewpoint of usage feeling.

Examples of polyols include ethylene glycol, propylene glycol, 1,3-butylene glycol, tetramethylene glycol, glycerin, sorbitol, diethylene glycol, dipropylene glycol, tetramethylene glycol, diglycerin, polyethylene glycol, and polypropylene glycol. Propylene glycol, dipropylene glycol, and 1,3-butylene glycol are particularly preferable. One or more water-based components selected from ethanol and polyols can be mixed in the range of 1 to 20% by mass, or 3 to 10% by mass with respect to the total amount of the cosmetic.

### <<Cosmetic Applications>>

As the cosmetic of the present invention is dried and concentrated, the usage feeling can change from a thick form to an elastic, gel-like form. Elastic gel-like cosmetics can also exhibit a refreshing feeling such that when a load exceeding a limit value is applied, the gel state collapses at once and water comes out. The cosmetic of the present invention can also be transparent or translucent, depending on the application.

The cosmetic of the present invention can be used, for example, in skin care cosmetics such as moisturizing gels, massage gels, beauty essences, beauty lotions, and emulsions; makeup cosmetics; sun care products; and hair cosmetics such as hair setting agents, hair gels; or hair dye.

### <<Cosmetic Production Method>>

The cosmetic of the present invention can be produced using a known method. For example, in the case of a cosmetic obtained by mixing a hydrophobically-modified polyether urethane into an oil-in-water emulsion having oil droplets, it can be prepared by preparing an oil-in-water emulsion having oil droplets by an aggregation method or a dispersion method as described above, diluting the emulsion with an water-based medium, such as ion-exchanged water, if necessary, and then adding the hydrophobically-modified polyether urethane solution dissolved in an appropriate amount of an water-based medium, if necessary.

### EXAMPLES

Hereinafter, the present invention will be described in more detail with reference to Examples, but the present invention is not limited thereto. Note that, hereinafter, unless otherwise specified, the mixing amounts are represented by mass %.

### Examples 1 to 7 and Comparative Examples 1 to 18

Regarding the cosmetics of the present invention obtained from the formulations by the production methods of Table 1 shown below and the cosmetics of the various commercial products A to R corresponding to Comparative Examples 1 to 18, evaluation of the viscosity at a shear rate of 1/s and the viscoelasticity ratio in a linear region was performed, and the data after five-fold concentration is shown in Table 2. These measurements were carried out under the conditions of 32° C. and 1 atm using a rheometer MCR302 manufactured by Anton Paar. Note that all of the cosmetics of Examples 1 to 7 prior to concentration had a viscoelastic ratio of 10 or more and a viscosity of 500 mPa·s or less at a shear rate of 1/s.

TABLE 1

| | Example 1 | Example 2 | Example 3 | Example 4 | Example 5 | Example 6 | Example 7 |
|---|---|---|---|---|---|---|---|
| Sodium Stearoyl | 0.6 | 0.6 | 0.6 | 0.48 | 0.54 | 0.48 | 0.54 |
| Glutamate¹⁾ | | | | | | | |
| Hydrophobically- | 0.1 | 0.2 | 0.3 | 0.2 | 0.2 | 0.3 | 0.3 |
| Modified | | | | | | | |
| Polyether Urethane²⁾ | | | | | | | |
| Ion-Exchanged Water | 86.80 | 86.70 | 86.60 | 88.12 | 87.41 | 88.02 | 87.31 |
| Dynamite Glycerine | 3 | 3 | 3 | 3 | 3 | 3 | 3 |
| Dipropylene Glycol | 3 | 3 | 3 | 3 | 3 | 3 | 3 |
| Phenoxyethanol | 0.3 | 0.3 | 0.3 | 0.24 | 0.27 | 0.24 | 0.27 |
| Methylparaben | 0.17 | 0.17 | 0.17 | 0.14 | 0.15 | 0.14 | 0.15 |
| Behenyl Alcohol | 0.99 | 0.99 | 0.99 | 0.79 | 0.89 | 0.79 | 0.89 |
| Stearyl Alcohol | 0.99 | 0.99 | 0.99 | 0.79 | 0.89 | 0.79 | 0.89 |
| Olefin Oligomer 30 | 4.05 | 4.05 | 4.05 | 3.24 | 3.65 | 3.24 | 3.65 |

¹⁾Amisoft (trademark) HS-11P (F) Manufactured by Ajinomoto Corp.
²⁾Adekanol GT-700 Manufactured by ADEKA Corp.

### <Cosmetic Production Method>

### Example 1

Olefin oligomer 30, behenyl alcohol, and stearyl alcohol were heated and dissolved at 80° C., and stirred and mixed to prepare a mixture A. While ion-exchanged water, sodium stearoyl glutamate, dynamite glycerin, dipropylene glycol, phenoxyethanol, and methyl paraben were heated and dissolved at 75° C. and stirred, mixture A was added thereto, and high-pressure emulsification was performed under a pressure of about 100 MPa to prepare an oil-in-water emulsion. A cosmetic was prepared by adding a hydrophobically-modified polyether urethane to the obtained oil-in-water emulsion, followed by stirring and mixing. The high-pressure emulsification was performed using a Nanomizer mark II high-pressure emulsifier (manufactured by Yoshida Machinery Kogyo Co., Ltd.) and an H-20 homogenizer (manufactured by Sanwa Engineering Co., Ltd.).

### Examples 2 to 7

The cosmetics of Examples 2 to 7 were prepared in the same manner as Example 1 except that the mixing ratios described in Table 1 were used.

TABLE 2

| | | Viscosity at | Viscoelastic |
|---|---|---|---|
| | | Shear Rate of | Ratio in Linear |
| | | 1/s (mPa · s) | Region (tanδ) |
| Example 1 | | 91800 | 0.128 |
| Example 2 | | 81300 | 0.293 |
| Example 3 | | 172000 | 0.00837 |
| Example 4 | | 123000 | 0.124 |
| Example 5 | | 77900 | 0.175 |
| Example 6 | | 81100 | 0.157 |
| Example 7 | | 138000 | 0.135 |
| Comp. Ex. 1 | Commercial Product A | 2840 | 0.504 |
| Comp. Ex. 2 | Commercial Product B | 7.4 | — |
| Comp. Ex. 3 | Commercial Product C | 1250 | 0.841 |
| Comp. Ex. 4 | Commercial Product D | 1670 | 0.172 |
| Comp. Ex. 5 | Commercial Product E | 8.09 | — |
| Comp. Ex. 6 | Commercial Product F | 8600 | 0.125 |
| Comp. Ex. 7 | Commercial Product G | 1020 | 1.05 |
| Comp. Ex. 8 | Commercial Product H | 10500 | 0.669 |
| Comp. Ex. 9 | Commercial Product I | 105 | — |
| Comp. Ex. 10 | Commercial Product J | 20000 | 0.418 |
| Comp. Ex. 11 | Commercial Product K | 45.5 | — |
| Comp. Ex. 12 | Commercial Product L | 30.5 | — |
| Comp. Ex. 13 | Commercial Product M | 5910 | 0.353 |
| Comp. Ex. 14 | Commercial Product N | 483 | — |
| Comp. Ex. 15 | Commercial Product O | 4710 | 0.416 |
| Comp. Ex. 16 | Commercial Product P | 3290 | 0.226 |
| Comp. Ex. 17 | Commercial Product Q | 11.2 | — |
| Comp. Ex. 18 | Commercial Product R | 29400 | 0.383 |

### <Results>

When the cosmetics of Examples 1 to 7 were applied to the skin, they produced a smooth, thick feeling immediately after application. However, as the cosmetics dried and were concentrated, they gradually changed to a gel-like form, producing a jiggly and elastic feeling. Such cosmetics did not produce a sticky feeling that is produced by cosmetics containing a high concentration of a hydrophobically-modified polyether urethane, and a moist feeling was obtained after drying and concentration. Conversely, in the case of the various cosmetics of Comparative Examples 1 to 18, even if these cosmetics were similarly applied to the skin and dried and concentrated, they did not change into a gel-like form.

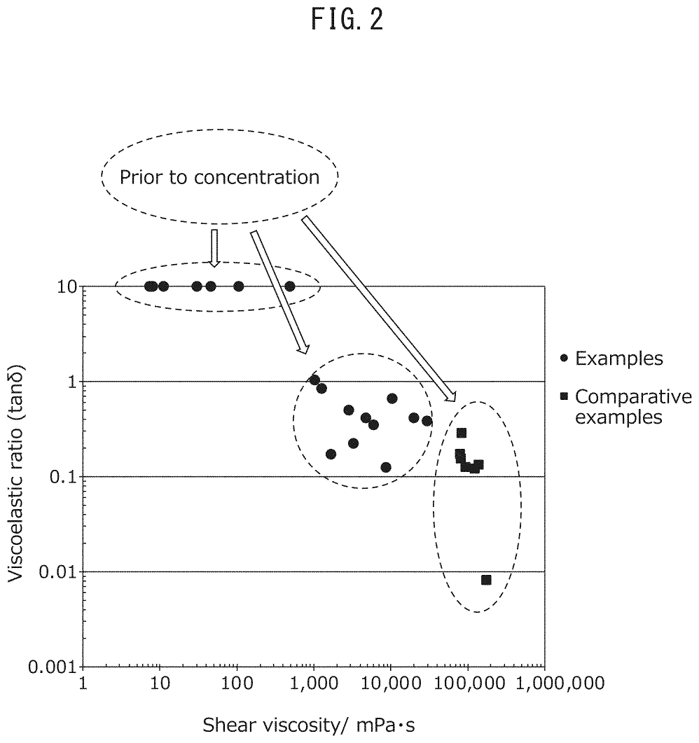

Subsequently, from the results of the viscoelasticity ratio and the viscosity at a shear rate of 1/s, it was attempted to examine the morphological changes of the cosmetics of Examples 1 to 7 in more detail. FIG. 2 relates to the cosmetics of Examples 1 to 7 and the various cosmetics of Comparative Examples 1 to 18 after five-fold concentration. The rough regions of viscosity and viscoelasticity ratio of the cosmetics of Examples 1 to 7 prior to concentration are schematically shown simultaneously, and when the concentrated viscoelastic ratio exceeded the measurement limit and could not be measured, the viscoelastic ratio was fixed at 10 for convenience of drawing the graph.

As is clear from the results of FIG. 2, it was confirmed that the differences in the morphological changes between the cosmetics of Examples 1 to 7 and the various cosmetics of Comparative Examples 1 to 18 could be clearly distinguished based on the results of the viscoelastic ratio and the viscosity at a shear rate of 1/s. Specifically, when the results of the viscosity and the viscoelasticity ratio prior to concentration of the cosmetics of Examples 1 to 7 were used as a reference, it could be confirmed that the cosmetics of Examples 1 to 7 changed greatly compared with the various cosmetics of Comparative Examples 1 to 18. From these results, it was discovered that when the viscosity at a shear rate of 1/s and viscoelastic ratio after five-fold concentration fall within predetermined ranges, as demonstrated in the cosmetics of Examples 1 to 7, further change from a thick form before concentration to a gel-like form with high elasticity can be quantified by such viscosity and viscoelastic ratio.

If a conventional cosmetic is highly concentrated to 10-fold or more or 20-fold or more, the same viscosity and viscoelastic ratio after concentration as those of the cosmetics of Examples 1 to 7 may be satisfied. However, it is clear from the data of Comparative Examples 1 to 18, which relate to various commercially available products, that there have been no cosmetics exhibiting such a morphological change by approximately five-fold concentration, i.e., such low-fold concentration that, when the cosmetics are applied to the skin, the morphological change can be actually felt. Further, such a novel usage feeling can be achieved if a specific viscosity and viscoelasticity ratio are satisfied, and it can be easily inferred that the same usage feeling can be exhibited as long as the viscosity and viscoelasticity ratio prior to and after concentration exhibit such a tendency, without being limited to the cosmetics of Examples 1 to 7.

### <<Cosmetic Formulation Examples>>

Examples of formulations of the cosmetic of the present invention are given below, but the cosmetic is not limited to these Examples. Note that the cosmetics described in the following formulation examples provided variable usage feelings based on the composition of the present invention, i.e., a thick, smooth feeling to a gel-like jiggly elastic feeling and a moist feeling after concentration.

<Formulation Example 1-Beauty Lotion>

| (Component) | (% by mass) |
|---|---|
| Purified Water | q.s. |
| Sodium Stearoyl Glutamate | 0.6 |
| Dynamite Glycerin | 3 |
| Dipropylene Glycol | 3 |
| Phenoxyethanol | 0.3 |
| Methylparaben | 0.17 |
| Behenyl Alcohol | 1 |
| Stearyl Alcohol | 1 |
| Olefin Oligomer 30 | 4.05 |
| Fragrance | q.s. |
| (PEG-240/Decyl Tetradeceth-20/HDI) copolymer | 0.3 |

### (Beauty Lotion Production Method)

Behenyl alcohol, stearyl alcohol, olefin oligomer 30, and fragrance were heated and dissolving at 80° C. and mixed to produce a mixture A. While ion-exchanged water, sodium stearoyl glutamate, glycerin, dipropylene glycol, phenoxyethanol, and methyl paraben were heated and dissolved at 75° C., and stirred, mixture A was added thereto, and high-pressure emulsification was performed under a pressure of about 100 MPa to prepare an oil-in-water emulsion. A (PEG-240/decyl tetradeceth-20/HDI) copolymer, which is a hydrophobically-modified polyether urethane, was added to the obtained oil-in-water emulsion and then stirred and mixed to prepare a beauty lotion. The high-pressure emulsification was performed using a Nanomizer mark II high-pressure emulsifier (manufactured by Yoshida Machinery Kogyo Co., Ltd.) and an H-20 homogenizer (manufactured by Sanwa Engineering Co., Ltd.).

1. A thick water-based cosmetic having a viscoelastic ratio of 10 or more and a viscosity of 500 mPa·s or less at a shear rate of 1/s, wherein
when concentrated by a factor of five by drying to remove moisture, the thick water-based cosmetic changes to a gel-like form having a viscoelastic ratio of 0.5 or less and a viscosity of 70000 mPa·s or more at a shear rate of 1/s.

2. The cosmetic according to claim 1, wherein the cosmetic in the gel-like form has a viscoelastic ratio of 0.2 or less and a viscosity of 75000 mPa·s or more at a shear rate of 1/s.

3. The cosmetic according to claim 1, wherein the cosmetic in the gel-like form has a viscoelastic ratio of 0.005 or more and a viscosity of 500000 mPa·s or less at a shear rate of 1/s.

4. The cosmetic according to claim 1, wherein in the cosmetic, an oil-in-water emulsion containing oil droplets is mixed with a hydrophobically-modified polyether urethane.

5. The cosmetic according to claim 4, wherein the average particle diameter of the oil droplets is 150 nm or less.

6. The cosmetic according to claim 4, wherein the oil droplets contain oil and a surfactant.

7. The cosmetic according to claim 4, wherein the hydrophobically-modified polyether urethan is represented by formula 1 below:
Rⁱ—{(O—Rⁱⁱ)ₖ—OCONH—Rⁱⁱⁱ[—NHCOO—(R^{iv}—O)ₚ—R^{v}]h}_{q} formula 1
where R¹, Rⁱⁱ, and R^{iv} each independently represent a C₂₋₄ hydrocarbon group,
Rⁱⁱⁱ represents a C₁₋₁₀ hydrocarbon group optionally having a urethane bond,
R^{v} represents a C₈₋₃₆ hydrocarbon group,
k is an integer of 1 to 500,
p is an integer of 1 to 200,
h is an integer of 1 or more, and
q is an integer of 2 or more.

8. The cosmetic according to claim 7, wherein the hydrophobically-modified polyether urethane is a polyethylene glycol-decyl tetradeceth-hexamethylene diisocyanate copolymer.
